SEQUENCE LISTING (21 sequences)

SEQ ID NO: 1 - protein, 5 aa, Unknown
  Mammalia
GLGHN

SEQ ID NO: 2 - protein, 6 aa, Unknown
  Mammalia
GLGHNQ

SEQ ID NO: 3 - protein, 7 aa, Unknown
  Mammalia
GLGHNQI

SEQ ID NO: 4 - protein, 8 aa, Unknown
  Mammalia
GLGHNQIR

SEQ ID NO: 5 - protein, 9 aa, Unknown
  Mammalia
GLGHNQIRM

SEQ ID NO: 6 - protein, 10 aa, Unknown
  Mammalia
GLGHNQIRMI

SEQ ID NO: 7 - protein, 11 aa, Unknown
  Mammalia
GLGHNQIRMIE

SEQ ID NO: 8 - protein, 12 aa, Unknown
  Mammalia
GLGHNQIRMIEN

SEQ ID NO: 9 - protein, 13 aa, Unknown
  Mammalia
GLGHNQIRMIENG

SEQ ID NO: 10 - protein, 14 aa, Unknown
  Mammalia
GLGHNQIRMIENGS

SEQ ID NO: 11 - protein, 15 aa, Unknown
  Mammalia
GLGHNQIRMIENGSC

SEQ ID NO: 12 - protein, 23 aa, Unknown
  Mammalia
  Cleavage site is at
RYSKLYRLGLGHNQIRMIENGSC

SEQ ID NO: 13 - protein, 19 aa, Unknown
  Mammalia
KLYRLGLGHNQIRMIENGS

SEQ ID NO: 14 - protein, 5 aa, Unknown
  Mammalia
KLYRL

SEQ ID NO: 15 - protein, 6 aa, Unknown
  Mammalia
SKLYRL

SEQ ID NO: 16 - protein, 7 aa, Unknown
  Mammalia
YSKLYRL

SEQ ID NO: 17 - protein, 8 aa, Unknown
  Mammalia
RYSKLYRL

SEQ ID NO: 18 - protein, 9 aa, Unknown
  Mammalia
LRYSKLYRL

SEQ ID NO: 19 - protein, 10 aa, Unknown
  Mammalia
LLRYSKLYRL

SEQ ID NO: 20 - protein, 11 aa, Unknown
  Mammalia
DLLRYSKLYRL

SEQ ID NO: 21 - protein, 12 aa, Unknown
  Mammalia
EDLLRYSKLYRL

(19) United States
(12) Patent Application Publication
(10) Pub. No.: US 2025/0271450 A1
(43) Pub. Date: Aug. 28, 2025

(54) BIGLYCAN PEPTIDE AND ANTIBODIES

(71) Applicant: SGPTH LIFE SCIENCE AB, KNIVSTA (SE)

(72) Inventors: Eva SKIÖLDEBRAND, GÖTEBORG (SE); Anders LINDAHL, ASKIM (SE); Stina EKMAN, KNIVSTA (SE)

(21) Appl. No.: 18/572,604

(22) PCT Filed: Jun. 23, 2022
(86) PCT No.: PCT/EP2022/067140

§ 371 Date: Dec. 20, 2023

(30) Foreign Application Priority Data
Jun. 23, 2021 (SE) 2150815-5

(51) Int. Cl. G01N 33/68; C07K 7/06; C07K 16/18; G01N 33/574

(52) U.S. Cl. CPC G01N 33/6893; C07K 7/06; C07K 16/18; G01N 33/57488; G01N 33/6878; G01N 2800/105

(57) ABSTRACT

There is provided an antibody that specifically binds to a peptide comprising the amino acid sequence N-terminal-GLGHN (SEQ ID NO 1), where the antibody binds to the sequence N-terminal-GLGHN (SEQ ID NO 1). The antibody can be used for diagnosis of for example bone sclerosis, fractures, chip fractures of the joint, avulsion fractures, bone bruise, osteoporosis, bone bruise, osteoporosis or cancer.

### CROSS-REFERENCE TO RELATED APPLICATIONS

This application claims priority to co-pending PCT International Application Serial No. PCT/EP2022/067140 filed Jun. 23, 2022, which claims priority to SE Patent Application No. 2150815-5 filed on Jun. 23, 2021, the entire content of both of which is incorporated herein by reference.

### SEQUENCE LISTING

The instant application contains a Sequence Listing which has been submitted electronically in ASCII format via EFS-Web and is hereby incorporated by reference in its entirety. Said Sequence Listing, created on Aug. 13, 2024, is named 47SGPT-NO10302PA corrected seq listing.txt and is 3,993 bytes in size.

### FIELD OF THE INVENTION

This invention relates to a peptide and antibodies against a peptide, and their use in diagnosis, in particular diagnosis of fractures, bone sclerosis and related diseases.

### BACKGROUND

Remodelling of the bone and generation of cartilage are two separate and carefully controlled physical phenomena. The two processes are regulated by different signalling mechanisms and are controlled by different cell types. For example, chondrocytes are responsible for remodelling of cartilage whereas osteoblasts and osteoclasts control bone remodelling and modelling. Bone changes, described as remodelling and modelling are seen simultaneously in many locations of the body. Bone is active metabolically throughout life and approximately 20% of all bone is replaced annually.

Bone modelling changes the shape and size of bone in response to physiologic and mechanical forces. This is most prominent in the growing animal. Bone remodelling in the adult animal, will replace and renew old bone matrix and maintain its strength and mineral homeostasis. Bone is well-vascularized and angiogenesis is involved in bone activity.

Cartilage, on the other hand, is not vascularised and has a very slow turnover with a very low renewal capacity.

The bone matrix consists of organic components such as collagen type I and versican and inorganic bone salts, mainly hydroxyapatite. In cartilage, collagen type II and aggrecan are the main matrix molecules. Biglycan is a small leucine-rich repeat proteoglycan which is expressed in both cartilage and bone connective tissue, that is believed to play a role in the mineralization of bone.

Remodelling of the bone may occur in different settings such as for example intense physical training, fractures and also in osteoarthritis.

Osteoarthritis (OA) is a low-grade chronic systemic inflammatory disease that results from breakdown of joint cartilage and underlying bone. The disease proceeds from an early phase which is characterized by inflammation and beginning disorganization of matrix proteins in the affected joints, to a more severe phase with damage to the underlying bone. The main symptom is pain in the joint. Osteoarthritis is a major clinical problem as it affects around 3.8% of the human population. It is estimated that 50% of NSAID pain killer prescriptions are related to OA.

Although joint pain often leads a physician to suspect OA, especially if the patient is elderly, it is today difficult to diagnose the early stages of OA. Today diagnosis is often based on identification, using radiology, of irreversible structural damages typical of the late stages of OA. X-ray or MRI cannot, however, be used to diagnose early-stage OA, because the structural damages are not yet visible. Also, X-ray and MRI examination requires expensive equipment, and making an appointment with a radiologist.

Osteoarthritis in horses is a great problem for horse owners. The principal reason for having a horse is to be able to use it, and often to use the horses in competitions. OA is the most frequent reason for not being able to use the horse due to lameness and OA accounts for the greatest single economic loss in the horse industry.

Outside the context of OA, remodelling of the bone occurs during the healing of bone fractures and after exercise. Exercise will cause the remodelling of the bone resulting in a subchondral bone sclerosis which is physiological at a certain level. However, the non-pathological bone sclerosis may cross over to a pathologic level, which will result in micro fractures in the interface of bone and cartilage.

Bone sclerosis is a condition characterized by hardening and increased density of the bone as the bone cells respond to mechanical load during training in the athlete or in the horse.

Microfractures are tiny fractures in a bone caused when the force applied to a bone exceeds the strength of that bone. This can be achieved through strenuous activities such as running, dance, military training or gymnastics. Hence, microfractures may arise from mechanical load. Microfractures may result in so-called chip fractures where a part of the bone and the cartilage detaches. Chip fractures are very problematic, in particular if they occur in the carpal joint of a horse. In many cases, the horse cannot recover from such an injury, additionally the chip fractures can proceed to catastrophic injury and the horse needs to be euthanized. In addition, a jockey can be severely injured when a gallop race horse receives a carpal fracture during a race. Chip fractures are also referred to as avulsion fractures.

Bone oedemas resulting from trauma to the bone may develop to bone sclerosis. Athletes often feel the urge to train and compete even if they feel a pain in the bone, for example a pain from a bone bruise. For example, soccer players frequently have such pain in their legs. Training with such pain may result in complications later, such as bone sclerosis or chip fractures. Therefore, it would be highly desirable to be able to monitor such conditions in order to be able to know how long an athlete should rest in order to prevent later complications.

Physiological bone sclerosis develops in racehorses during training, which can eventually progress to pathology with presence of micro fractures leading to chip fractures and acute catastrophic intra-articular fractures (Diab et al. J Vet Diagn Invest. 2017; 29 (4): 405-413).

The remodelling of the bone in the OA-affected joint may also result in micro fractures in the bone. These micro fractures may, in turn, result in so-called chip fractures where a part of the bone and the cartilage detaches. Chip fractures are very problematic, in particular if they occur in the carpal joint of a horse. In many cases, the horse cannot recover from such an injury, additionally the chip fractures can proceed to catastrophic injury and the horse needs to be euthanized.

OA in horses and humans are caused by similar mechanisms. In both humans and horses OA proceeds from an early stage characterized by inflammation over months and years to a later stage with extensive tissue damage (Goldring, M. B. and Otero M. Current Opinion in Rheumatology (2011) 23 (5): 471). OA disease mechanisms in humans and horses are also very similar on the molecular level (Stenberg J, Rüetschi U, Skiöldebrand E, Kärrholm J, Lindahl A. Proteome Sci. (2013) October 4; 11 (1): 43) (Svala E, Löfgren M, Sihlbom C, Rüetschi U, Lindahl A, Ekman S, Skiöldebrand E. Connect Tissue Res. (2015); 56 (4): 315-25). Fatigue-related changes in the subchondral bone (SCB) microenvironment, is one part of the osteoarthritis process (Hu et al Bone Res. 2021; 9 (1): 20). This typically happens before onset of damages to the cartilage during OA. WO 2017216289 discloses the use of a fragment of COMP for the diagnosis of OA. Fragment of COMP is a biomarker of cartilage degradation.

It would be useful if there were more convenient ways of diagnosing and staging OA. The lack of early markers for OA hampers development of drugs that could be used to control the disease before it manifests as irreversible tissue damage. Hence, there is still a need for improved biomarkers for OA.

Currently there are no diagnostic tools that can identify the early stages of bone remodelling eventually leading into micro fractures causing pain and subsequent lameness in the horse.

There is a need for preventing and diagnosing and preventing microfractures and bone sclerosis and over training and associated conditions. In addition, there is a need for improved methods for diagnosing cancer, in particular colon cancer.

Diagnosis is often carried out using blood samples. This may cause discomfort and is not completely without risk for the patients as it involves drawing a blood sample from a vein. It would be useful if such diagnosis could be carried out in a more convenient manner.

The ground material for riding arenas (so called “footings”) are very important for the health of the horse. Too hard footings, for example, may lead to increased subchondral bone and fractures. New footings have been developed recently. For example, a mixture of sand and polymer fibers have been introduced in recent years. There is some uncertainty as to the effect of new footings on the health of the horse. There is a need for better methods to investigate such footings.

This invention solves these and other problems.

### SUMMARY OF THE INVENTION

The inventors have surprisingly found shown that a cleavage fragment of biglycan (BGN²⁶²) (N-terminal-GLGHN (SEQ ID NO 1)) is prominent in bone destruction and subchondral bone sclerosis, and other disease.

In a first aspect of the invention there is provided an antibody that specifically binds to a peptide comprising the amino acid sequence N-terminal-GLGHN (SEQ ID NO 1).

The antibody can be used for the detection of a proteolytic fragment of biglycan that exposes the N-terminal.

In a second aspect of the invention there is provided the antibody according to the first aspect of the invention for use in diagnosis. The diagnosis may be diagnosis of osteoarthritis, bone sclerosis, fractures, chip fractures of the joint, avulsion fractures, bone bruise, osteoporosis, cancer, atherosclerotic plaques, aortic valve stenosis, Kashin-Beck disease, tendinitis, eye disorders, skin disorders, fibrosis, rheumatoid arthritis, lupus nephritis, diabetes, calcified aortic valve disease, perimyocarditis, insulin-dependent diabetes mellitus type 1, or Crohn's disease in a subject.

In particular, the diagnosis may be diagnosis of osteoarthritis, bone sclerosis, fractures, avulsion fractures, chip fractures of the joint, bone bruise or osteoporosis.

In a preferred embodiment the antibody is used for detecting the amount of a peptide comprising the amino acid sequence N-terminal-GLGHN in a sample from a subject. The sample may be any suitable sample, for example a sample of synovial fluid, spinal fluid (liquor), serum, blood, blood plasma, urine or saliva.

In a third aspect of the invention there is provided a method of diagnosis comprising isolating a sample from a subject and analysing the sample for presence of a peptide comprising the amino acid sequence N-terminal-GLGHN in the sample.

In a fourth aspect of the invention there is provided a method for diagnosis of a disease selected from bone sclerosis, fractures, chip fractures of the joint, avulsion fractures, bone bruise, osteoporosis, cancer, atherosclerotic plaques, aortic valve stenosis, Kashin-Beck disease, tendinitis, eye disorders, skin disorders, fibrosis, rheumatoid arthritis, lupus nephritis, diabetes, calcified aortic valve disease, perimyocarditis, insulin-dependent diabetes mellitus type 1, or Crohn's disease comprising providing a sample previously isolated from a subject and analysing the sample for presence of a peptide comprising the amino acid sequence N-terminal-GLGHN in the sample.

In preferred embodiments the disease is selected from one or more of bone sclerosis, fractures, chip fractures of the joint, avulsion fractures, bone bruise, osteoporosis or cancer, in particular bone sclerosis, fractures, chip fractures of the joint, avulsion fractures, bone bruise or osteoporosis. In one embodiment the disease is cancer, in particular colon cancer.

The sample may be one of synovial fluid, spinal fluid (liquor), serum, blood, blood plasma, urine or saliva, in particular saliva.

In yet another aspect of the invention there is provided a method for preventing osteoarthritis, bone sclerosis, fractures, chip fractures of the joint, avulsion fractures, bone bruise, osteoporosis in a subject to comprising the steps of:
a) obtaining a sample from the subject and analysing the presence of a peptide comprising the amino acid sequence N-terminal-GLGHN (SEQ ID NO 1) in the sample,
b) if the level of peptide in the subject in a sample is above a predetermined level, determining that the subject should be treated, where the treatment is resting the subject.

In a different aspect of the invention there is provided a method for preventing osteoarthritis, bone sclerosis, fractures, or chip fractures of the joint in a subject to comprising the steps of:
a) repeatedly obtaining samples from the subject and analysing the presence of a peptide comprising the amino acid sequence N-terminal-GLGHN (SEQ ID NO 1) in the samples,
b) if the level of peptide in the subject in a sample is above a predetermined level, determining that the subject should be treated, where the treatment is resting the subject.

In a different aspect of the invention there is provided a kit comprising an antibody according to the first aspect of the invention. The antibody may be comprised in a diagnostic device for single use, where the kit additionally comprises a saliva sampling device. The diagnostic device may be a lateral flow device.

In a different aspect of the invention there is provided a peptide comprising the amino acid sequence N-terminal-GLGHN (SEQ ID NO 1).

In a different aspect of the invention there is provided the use of a peptide comprising an amino acid sequence N-terminal-GLGHN (SEQ ID NO 1) for the production of an antibody.

In yet a different aspect of the invention there is provided a method for determining the properties of a footing for a horse arena, comprising obtaining a sample from a horse that has been exercising on the footing and analysing the sample for presence of a peptide comprising the amino acid sequence N-terminal-GLGHN in the sample.

### BRIEF DESCRIPTION OF DRAWINGS

FIG. 1 is a diagram that shows ELISA data, showing the specificity of the antibody.

FIG. 2 are images of immunohistochemistry staining of biglycan neo-epitope of cartilage and bone sections from carpal joints from horses.

FIG. 3 is a diagram that shows ELISA data from horse synovial fluid.

FIG. 4 is a diagram that shows ELISA data from synovial fluid.

FIG. 5 is a diagram that shows ELISA data from horse serum.

FIG. 6 is a diagram that shows the presence of biglycan neoepitope in saliva from horses.

FIG. 7 is a diagram that shows the presence of biglycan neoepitope in healthy horses that undergo training.

FIG. 8 is a diagram that shows the presence of biglycan neoepitope in cancer patients.

FIGS. 9-10 shows the presence of biglycan neoepitope in saliva from horses.

FIGS. 11-12 show presence of biglycan neo-epitope in saliva from humans.

### DETAILED DESCRIPTION

This invention relates to a peptide comprising a cleavage fragment of biglycan, in particular the sequence N-terminal-GLGHN (SEQ ID NO 1) (“biglycan neoepitope”), an antibody against this peptide and the use of such an antibody in diagnosis.

The peptide may have a length of from 5 to 100, preferably from 5 to 30, more preferably from 5 to 20 amino acids, more preferably from 5 to 9 amino acids, as long as the N-terminal has the sequence GLGHN. The first glycine residue of this sequence of the peptide thus has the NH₂ group of the peptide. The peptide may have a length of at least 5 amino acid residues, more preferably at least 6 amino acid residues, more preferably at least 7 amino acid residues and most preferably at least 8 amino acid residues.

In one embodiment the peptide has a length such that it can be used for immunization.

The peptide may be an isolated peptide. The peptide may be isolated from for example synovial fluid, blood, plasma or serum. The peptide may also be synthesized, using methods known in the art, for example R. B. Merrifield (1963). “Solid Phase Peptide Synthesis. I. The Synthesis of a Tetrapeptide”. J. Am. Chem. Soc. 85 (14): 2149-2154 and Schnolzer, M. A., P.; Jones, A.; Alewood, D.; Kent, S. B. H. (2007). “In Situ Neutralization in Boc-chemistry Solid Phase Peptide Synthesis”. Int. J. Peptide Res. Therap. 13 (1-2): 31-44.

The peptide can be used for the production and isolation of an antibody. The antibody specifically binds to a peptide comprising or consists of the amino acid sequence N-terminal-GLGHN. The term “antibody” also includes Fab, Fab′, F(ab′) 2, Fv, and single-chain antibodies and similar types of proteins that binds to an epitope with a high specificity, in particular proteins that are derived from or comprises a fragment from an antibody, in particular proteins that comprises a variable chain from an antibody that binds to N-terminal GLGHN. Methods for producing antibodies against a peptide are well known. The peptide can be used for screening for antibodies that bind to the peptide and also for purifying the antibody.

Preferably the antibody has a high affinity for the peptide. Affinity can be expressed using the dissociation constant (Kd). Preferred binding affinities include those with a dissociation constant (Kd) less than 10⁻⁶ M, more preferably 5×10⁻⁷ M, more preferably 10⁻⁷ M, more preferably 5×10⁻⁸ M, more preferably 10⁻⁸ M, more preferably 5×10⁻⁹ M, more preferably 10⁻⁹ M, more preferably 5×10⁻¹⁰ M, more preferably 10⁻¹⁰ M, more preferably 5×10⁻¹¹ M, more preferably 10⁻¹¹ M, more preferably 5×10⁻¹² M, more preferably 10⁻¹² M, even more preferably 5×10⁻¹³ M, or and most preferably less than 10⁻¹³ M. Preferably the antibody is an isolated antibody. The antibody may be a purified antibody.

Preferably the antibody binds specifically to a peptide comprising or consist of the sequence N-terminal-GLGHN (SEQ ID NO 1), in particular N-terminal-GLGHNQ (SEQ ID NO 2), more preferred N-terminal GLGHNQI (SEQ ID NO 3) and most preferred N-terminal GLGHNQIR (SEQ ID NO 4), N-terminal-GLGHNQIRM (SEQ ID NO 5), N-terminal-GLGHNQIRMI (SEQ ID NO 6), N-terminal-GLGHNQIRMIE (SEQ ID NO 7), N-terminal-GLGHNQIRMIEN (SEQ ID NO 8), N-terminal-GLGHNQIRMIENG (SEQ ID NO 9), N-terminal-GLGHNQIRMIENGS (SEQ ID NO 10) or N-terminal-GLGHNQIRMIENGSC (SEQ ID NO 11). For generating the antibody, it may be suitable to immunize with a peptide that is longer than N-terminal-GLGHN, for example the immunisation peptide GLGHNQIRMIE (SEQ ID NO 7).

In various embodiments of the invention, the sequence upstream of the biglycan cleavage site is used. These peptides and antibodies against these peptides are used in the same manner the other peptides described herein. In particular, a peptide with the sequence KLYRL-C-terminal (SEQ ID NO 14), more preferably SKLYRL-C-terminal (SEQ ID NO 15), more preferably YSKLYRL-C-terminal (SEQ ID NO 16), more preferably RYSKLYRL-C-terminal (SEQ ID NO 17), more preferably LRYSKLYRL-C-terminal (SEQ ID NO 18), more preferably LLRYSKLYRL-C-terminal (SEQ ID NO 19), more preferably DLLRYSKLYRL-C-terminal (SEQ ID NO 20), and most preferably EDLLRYSKLYRL-C-terminal (SEQ ID NO 21) may be detected.

The antibody may be any antibody derived from a mammal such as mouse, rat, hamster, rabbit, goat, horse or chicken, and the like, among which mouse is preferred. The isotype of the antibodies may be any of IgG, IgM, IgE, IgA, IgY and the like.

The antibody may be a polyclonal antibody, produced by immunisation of an animal, for example a rabbit, as is known in the art. But preferably the antibody is a monoclonal antibody. Preferably the monoclonal antibody is a mouse or rabbit monoclonal antibody.

There are well-known methods for producing, purifying and isolating antibodies and determining their binding capacity. There are also well-known methods for using an antibody to determine the presence of an antigen. It is referred to Current Protocols in Immunology and Current Protocols in Molecular Biology for details.

Monoclonal antibodies against the peptide may be generated using the well-known hybridoma technology (Kohler and Milstein, Nature, 256, 495-497, 1975). A single clone can be isolated by limiting dilution analysis, the soft agar assay, a method using a fluorescence activated cell sorter and the like. In the limiting dilution analysis, for example, colonies of the hybridoma are serially diluted to around 1 cell/well in a medium before cultivation to isolate the hybridoma which produces the desired antibody. The antibody may be a chimeric antibody or a humanized antibody.

Antibody clones may also be generated using other methods, such as, for example, phage display.

When the antibody is murine IgG, the antibody can be purified with affinity chromatography using a Protein A-conjugated carrier or an anti-mouse immunoglobulin-conjugated carrier.

The antibody can be used for diagnosis in different manners. The antibody can be used for measuring the presence, the amount of or concentration of the peptide in a sample from a subject, which may be a human or an animal. The antibody may be contacted with a sample from a subject. The sample may be any type of biological sample, for example synovial fluid, blood, saliva, plasma, serum, spinal fluid (liquor) or urine, ascites, or biological tissues used in histological section. Examples of useful tissues for sections include bone, cartilage tendon, eye, pancreas, aorta, kidney and skin. Preferably the sample is a liquid sample. In a preferred embodiment the sample is a serum sample, a blood sample, a plasma sample or a sample of synovial fluid. In one even more preferred embodiment the sample is a sample of synovial fluid. In one preferred embodiment the sample is a urine sample or a saliva sample, in particular a saliva sample. A sample of a suitable volume is collected. When the sample is in liquid form, in particular a saliva sample or a synovial fluid sample, the sample may for example have a volume of from 50 ul-2000 ul. A saliva sample is suitable at least 300 ul, more preferably at least 500 ul. When the sample is a saliva sample it may be useful to rest the subject before the sample is taken. The resting period may be for example at least 30 minutes, more preferably at least 1 hour. It may also be useful to compare the measure the peptide level when resting with the peptide level immediately after exercise.

The sample may be isolated from a subject. The sample may be isolated from the subject before the binding step is carried out. Hence the method of diagnosis may comprise the step of providing a sample that has previously been isolated from a subject. The diagnosis method may be carried out in vitro.

A convenient manner to measure the concentration of a peptide in a sample with an antibody is ELISA. The design and use of ELISA (enzyme-linked immunosorbent assay) is well known in the art of diagnostics. The antibody can also be used in, for example, immunohistochemistry. For example, thin sections of tissue, for example tissue that is frozen, paraffin-treated or fixed, may be stained using the antibody as is known in the arts of histopathology. The antibody can also be used in western blot or Wes or Simple Western systems.

The antibody can be detected in various manners. A frequently used method is to use a secondary antibody that is conjugated with a substance that can be detected (a marker or label), for example an enzyme (such as HRP), a fluorophore or a radiolabel. For example, if the primary antibody is a mouse antibody, the secondary antibody can be a goat anti-mouse antibody. The presence of the marker can be detected with methods known in the art: an enzyme may be detected with reagents that produces a colour or light, a radiolabel may be detected with a scintillator or photographic film, and a fluorophore may be detected with a fluorescence detector or viewed in a fluorescence microscope.

Alternatively, the primary antibody (anti-N-terminal-GLGHN-antibody) may be directly conjugated with a marker/label.

A suitable working concentration of the antibody when it is used in various procedures such as ELISA or immunohistochemistry depends on the affinity of the antibody and can be determined by testing different concentrations of the antibody in order to find a concentration that gives a good signal to noise ratio. As an example, an antibody stock with a concentration of 1 mg/ml may be diluted at 1/100, 1/200, 1/1000 and 1/5000 for testing a suitable working concentration. Working concentrations of the antibody in these procedures is usually in the range of μg/ml, for example 1 ng-10 μg/ml. The antibody is suitable diluted in PBS, possibly with the use of an additional protein such as BSA and a preservative, such as sodium azide.

The antibody can be used for diagnosis of a disease in a subject, in particular in a human or in a horse. However, the subject may also be a, a cow, a dog, a cat, a sheep, a pig, a rat or a mouse or any other mammal. For example, the concentration of the peptide in a sample can be determined using standard methods, for example ELISA. The thus determined concentration can be compared against a standard value (reference value) or a cut-off level. A deviation from the standard value may be indicative of a particular disease, or a stage of the disease.

In various embodiments, the risk for developing a condition is determined. The subject may be suspected of having a condition. Presence of the peptide in the sample may be indicative of the condition or a risk for the condition.

The diagnosis may be diagnosis of a disease associated with systemic inflammation in particular a systemic low-grade chronic inflammation. The presence of the peptide indicates a disease. The diagnosis may thus be diagnosis of osteoarthritis, bone sclerosis, fractures, avulsion fractures, chip fractures of the joint, bone bruise, osteoporosis, cancer, atherosclerotic plaques, aortic valve stenosis, Kashin-Beck disease, tendinitis, eye disorders, skin disorders, fibrosis, rheumatoid arthritis, lupus nephritis, diabetes, calcified aortic valve disease, perimyocarditis, insulin-dependent diabetes mellitus type 1, or Crohn's disease in a subject. In a preferred embodiment, the diagnosis is diagnosis of osteoarthritis, bone sclerosis, fractures, avulsion fractures or chip fractures of the joint, or bone bruise or osteoporosis.

In various embodiments, the diagnosis is diagnosis of cancer, in particular colon cancer. Other cancers that can be diagnosed includes lung cancer, liver cancer, kidney cancer, bladder cancer, pancreatic cancer, gastric cancer, kidney cancer, breast cancer, cervix cancer, skin cancer, prostate cancer and endometrial cancer. Presence of the peptide N-terminal-GLGHN correlates with invasive properties of the tumour.

In various preferred embodiments the diagnosis is diagnosis of one or more of bone sclerosis, fractures, avulsion fractures, chip fractures of the joint. Presence of the peptide may be used for monitoring the proceeding from physiological to pathological bone sclerosis, progression to micro fractures, progression to chip fractures or avulsion fractures and progression to catastrophic injury (intra-articular fractures).

The antibody can be used for diagnosis of OA, in mammals, in particular in humans or in horses. In a preferred embodiment, high concentration of the peptide in synovial fluid or other sample may indicate early OA such as for example OA with early osteochondral lesions, subchondral remodelling, or osteochondral splitting. Low concentration of the peptide may indicate no OA. Presence of the peptide may be used for monitoring the proceeding from physiological to pathological bone sclerosis, progression to micro fractures, progression to chip fractures and progression to catastrophic injury (intra-articular fractures). The peptide may also be used to monitor acute lameness/early OA to chronic lameness/chronic OA, in particular in horses.

The cut-off levels may depend on the method used and may be established using standard experiments and appropriate controls. For example, the level of the peptide in a sample of subjects with the disease (where the disease is determined in a different manner than using the peptide) is compared with the level of peptide in a group of healthy subjects. Hence the cut-off value may be predetermined. The cut-off value may be for example 150%, more preferably 200%, and most preferably 300% of the of the average level for healthy subjects. A cut-off value for serum or synovial fluid may be 300 ng/ml, more preferably 500 ng/ml, more preferably 600 ng/ml more preferably 800 ng/ml and most preferably 1000 ng/ml. A cut-off value for saliva may be 20 ng/ml, more preferably 100 ng/ml and more preferably 500 ng/ml and most preferably 1000 ng/ml. However, suitable cut-off values are to be selected on the condition that is being diagnosed, the species of the subject and the type of sample.

When the method has been used to diagnose osteoarthritis, bone sclerosis, fractures or chip fractures of the joint, or the risk for developing such conditions, the subject may be treated.

One form of treatment, in particular for horses, is to rest the subject. For example, the amount of training is reduced. Furthermore, when the subject is a human, in particular an athlete, the treatment may be to reduce training or rest.

In one embodiment a subject is screened for high amount of peptide. For example, samples are repeatedly collected and analyzed with a time interval which may be at least 3 days, more preferably at least seven days, more preferably at least 1 month and most preferably at least 3 months. The time interval may have an upper limit, for example from 7 days to 6 months. For example, as a preventive measure, the peptide can be detected in saliva, serum or synovial fluid in horses subject to training. In case of increased concentration of peptide in horse, in particular a non-lame horse, one treatment option is to decrease the amount of training. The training can be kept at low intensity for a period until the peptide level in synovial fluid and serum has gone down again. A baseline peptide level may be determined for the subject when the subject is healthy. A cut-off value may be used. In this way, the optimal amount of training for a subject may be established.

As an additional step, in case of a lame horse with high concentration of biglycan fragment in serum or synovial fluid, arthroscopy may be used as an additional diagnostic modality. This may be followed by adequate treatment. Pharmacological treatment for example the treatment described in WO 2020/084113 (sildenafil treatment). If there is a chip-fracture, the fragment may be removed during arthroscopy. During rehabilitation, the biomarker can be monitored to decide the amount of training allowed.

The antibody and the necessary reagents may be included in a kit for detecting the peptide in a sample. The kit may be based on ELISA, for example competitive ELISA. The kit may include a stationary phase (such as a plate with wells), secondary antibodies, peptides, buffers and reagents for detecting the marker or label.

In a preferred embodiment the kit comprises a sample collection device, in particular a saliva collection device, for example a saliva collection device for horses. A saliva collection device for human subjects may comprise a test tube with a funnel for a subject to spit a saliva sample into. A saliva collection device for horses or other animals may comprise a handle for holding the device connected to a swab. The user holds the handle and inserts the swab into the mouth of the subject, which may be a horse. The swab then absorbs a sample of saliva. After collection, the swab can be inserted into a sample tube for later analysis. Examples include the EquiSal saliva collection swab by Austin Davies Biologics Ltd. The sample collection device may comprise a buffer for preserving the sample.

The kit may comprise a diagnostic device for single use for example a lateral flow device which incorporates the antibody. Such lateral flow devices are known and typically comprise an antibody that specifically bind to an antigen of interest, and a detection means, for example reagents that generate a colour or a fluorescent signal or other signal, if the antibody binds to the antigen. The presence of a colour or fluorescent signal indicates the presence of antigen in the sample. The lateral flow device typically comprise means for determining the presence of a control substance in the sample (positive control). Examples of suitable lateral flow test include the ones disclosed in U.S. Pat. Nos. 6,485,982 and 9,034,657 and references cited therein.

In some embodiments the amount of antigen (in this case the peptide) can be quantified by reading a colorimetric or fluorescent signal, for example from lateral flow device. A reader may be arranged to read a colorimetric or fluorescent signal and quantify the signal. In this way a level of peptide in sample can be determined and compared to a cut-off value. A suitable reader for a diagnostic device may be the Reusable Reader from Lumos Diagnostics Ltd.

Diagnosis may also be carried out by detecting the peptides described herein, such as N-terminal-GLGHN and KLYRL-C-terminal, by other means than an antibody. Suitable methods include sequencing peptides by mass spectrometry and peptide sequencing by Edman degradation.

The peptide or antibody may also be used for determining the properties of a footing for a horse arena. A softer footing will generate less stress on the joint of the horse and will result in a lower amount of the peptide in a sample from the horse. Hence a method for determining the properties of a footing for a horse arena may comprise obtaining a sample from a horse that has been exercising on the footing and analysing the sample for presence of a peptide comprising the peptide in the sample. Result from different footings may be compared in this manner. Preferably a number of healthy horses, for example from 3 to 10 horses, are allowed to exercise on the selected footings for a predetermined time and samples, for example serum samples or saliva samples, are collected from each horse after a predetermined time period. Preferably each horse is allowed to rest between testing two different footings so that the peptide levels are allowed to come back to a resting level before the next trial. A baseline level may be determined for each horse when the horse is resting.

### Example 1

The biglycan cleavage site RYSKLYRL*GLGHNQIRMIENGSC (SEQ ID NO 12) where the * indicates the cleavage site is located 258 amino acids from the N-terminal of native horse biglycan (UniProtKB/Swiss-Prot: 046403.1).

A monoclonal antibody against the amino acid peptide GLGHNQIRMIE (SEQ ID NO 7) was produced (Genescript) in rabbits. Furthermore, a specific polyclonal antibody against the peptide GLGHNQIRMIE was a produced in rabbits.

Bioinformatics analysis revealed the amino acid sequence around the cleavage site (RYSKLYRL*GLGHNQIRMIENGSC) is completely conserved in all analysed mammal species (human, horse, cow, pig, mouse, rat, sheep, rabbit, dog and domestic cat) (Data not shown). Hence it can be expected that the antibody will be useful for detecting the neoepitope in all mammals.

### Example 2

An inhibitory ELISA was developed and evaluated for detection of the biglycan neoepitope in horse serum using the peptide GLGHNQIRMIENGSC (Big Neo) (SEQ ID NO 11) also referred to as BGN²⁶² or “biglycan neoepitope” The freeze-dried peptide was reconstituted according to the Genscript peptide solubility guidelines. In short, the Big Neo peptide was reconstituted in distilled water to a concentration of 1 mg/mL and thereafter aliquoted, frozen and stored in −80° C. until use.

The inhibitory ELISA started with coating the plate with Big Neo. Using Nunc MaxiSorp™ Clear Flat-Bottom 96-Well Plates (Invitrogen) and the addition of Big Neo (100 μL/well, Genscript) diluted to 1 μg/mL in 100 mM carbonate buffer with pH 9.6, the peptide was coated over night at 4° C., denoted as the ELISA-plate.

The calibration standard was prepared from the stock of Big Neo peptide (1 mg/mL). First the highest standard point was set at 2000 ng/mL with a dilution in MultiBooster (Kementec) and thereafter using 11 step-1:2 serial dilution (1 mL peptide+1 mL MultiBooster) the calibration standard was made ranging from 0 (the 11th with no peptide) to 2000 ng/mL.

Serum samples was also prepared by dilution 1:20 and synovial fluid diluted 1:4 in Multibooster (Kementec). Serum dilution was determined after analyzing the serial dilution of normal serum where the primary antibody found the most peptide at 1:20 dilution. As serum control we used Equidae serum (lot.2109875, Gibco).

The monoclonal antibody of Example 1 (0.681 mg/mL) was used as primary antibody. The primary antibody was diluted in MultiBooster to a concentration of 30 ng/mL.

100 μL of each concentration of calibration standard and samples (in duplicates) was added to Thermo Scientific™ Sterilin™ Clear Microtiter™ Plates (Fisher Scientific). The 30 ng/ml diluted primary antibody (100 μl/well) was added to each standard as well as samples and thereafter pre-incubated overnight in humid chamber within a rotation incubator (39 rpm) with temperature set at 37° C.

Overnight, after 17 hours, the coated ELISA-plate was washed 4 times in the wash buffer (10 mM PBS with 0.05% Tween, pH 7.4) using Tecan Hydro wash and thereafter blocked with synthetic blocker (Kementec) for 0.5 hour at 37° C. After blockage the pre-incubated standards and samples (50 μL/well) were transferred to the ELISA-plate and incubated for 1 hour at room temperature on the ELISA-plate shaker set at 600 rpm. After the 1-hour incubation the ELISA-plate with primary antibody, standard and samples was washed 4 times in wash buffer. The secondary polyclonal goat anti rabbit (IgG) HRP 1 mg/mL (Abcam) was diluted 1:50 000 in 10 mM PBS with 0.05% Tween and 0.1% BSA, pH 7.4. Then, 50 μL/well of the secondary antibody was added to the standard and sample wells in the ELISA-plate and incubated in the dark for 30 minutes on the ELISA-shaker set at 600 rpm. Thereafter the ELISA-plate was washed again, this time 8 times in wash buffer. Next TMB was added, 50 μL/well, and incubated in the dark at RT and stopped after 20-30 minutes with 0.18M H2SO4. Absorbance was evaluated at 450 nm and scanned in SPARK multifunctional plate reader using Magellan software (Tecan).

To evaluate the specificity of the primary antibody we used an overlapping control peptide (OL) with the sequence KLYRLGLGHNQIRMIENGS (SEQ ID NO 13) as coating peptide and as antigen in the preincubation were we made series dilution such as the calibration standard.

OL control peptide:
(SEQ ID NO 13)
KLYRLGLGHNQIRMIENGS
Big Neo peptide:
(SEQ ID NO 11)
GLGHNQIRMIENGSC

The intra-assay precision was investigated within the inhibitory Big Neo ELISA. The Equidae control serum were used in 6 replicates. The inter-assay variation was also examined for the Equidae control serum as 6 replicates in a total of 3 assays on different occasions. The lowest and highest detections level was investigated during one ELISA with n=6 replicates of the standard curve points and a CV calculation of the replicates below 20% was granted as detectible values. An assay of spike and recovery was also done using 5 different samples with spiked 125 ng/ml medium for dilution 1:20 or normal multibooster for dilution 1:20. The recovery was thereafter calculated from the concentration obtained in the spike minus the concentration of the 1:20 diluted sample.

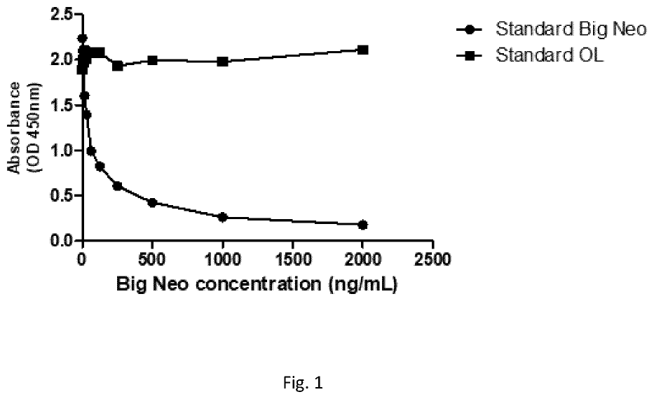

The specificity of the primary antibody against Big Neo and the overlap peptide (OL) was tested. Both peptides were serial diluted as calibration standards ranging from 0-2000 ng/mL. The monoclonal antibody showed high specificity for Big Neo. The epitope was detectable in serum. We verified that the monoclonal antibody could not detect the OL peptide (FIG. 1). This shows that the N-terminal of the epitope was crucial for antibody binding.

The lowest and the highest detection level of Big Neo were 1.95 and 2000 ng/ml and the CV (%) within one assay, the intra-assay were 5.5% and the between inter-assay CV were 4.3%.

With the 125 ng/ml spiked serum from 5 different individuals we recovered a mean of 120 ng/ml and with a 12% CV.

### Example 3

Osteochondral samples from the third carpal bone, were immediately (within 1 hour after slaughter) immersed in 10% neutral buffered formalin. The tissues were trimmed into 5 mm thick slabs, with a band saw (Exact 312 Diamond Band Saw, Exact Technologies, Inc. Oklahoma City, USA), dehydrated, embedded in paraffin, decalcified in 3.4% (w/v) sodium formiate and 15.1% (v/v) formic acid, cut into 4-5 μm sections and stained with hematoxylin and eosin (H&E) for microscopic examination. Sections were also deparaffinazed, rehydrated and prepared for immunostaining. Non-specific staining was blocked with 3% H2O2 in 10 mM PBS with 0.05% Tween, pH 7,4 at for 5 min in RT. Immunostaining for native biglycan and the biglycan neo-epitope was done at 4° C. overnight with a rabbit polyclonal anti-equine biglycan antibody (Lot. A117664, Invitrogen) in a dilution 1:750) and the polyclonal antibody against the equine biglycan neo-epitope (0.mg/mL) in a dilution of 1:4000) in 10 mM PBS with 0.05% Tween, pH 7.4.

The sections aimed at visualizing the native biglycan were pretreated with hyaluronidase (1 mg/mL in PBS) for 1 hour in 37° C., and directly incubated with chondrotinase (0.05 U/mL) for 1 hour in 37° C.

As controls, Rabbit immunoglobulin fraction (#X0903 Lot: 20066518 Dako Denmark A/S) for native biglycan and Recombinant rabbit IgG, monoclonal ([EPR25A]—isotype control #ab 172730 Lot GR3235749-24 (Abcam, United states) for the neo-epitope were used instead of the primary antibodies. The same protein concentration as the primary antibodies were used for the isotypes.

Staining was visualized using the HRP conjugated anti rabbit En Vision (DAKO) for 30 min. and the color developer 3,3-diaminobenzidine, DAB.

The osteochondral samples were evaluated microscopically using the recommended assessments for OA in horse (McIlwraith et al 2010). The scoring includes the articular cartilage, the cartilage bone interface and the underlying sub chondral bone. Microscopic assessments of articular cartilage (0-16) as: chondrocyte necrosis (0-4), cluster formation (0-4), fissuring (0-4), and focal cell loss (0-4) and osteochondral area (0-10) as osteochondral lesions (0-4), subchondral remodeling (0-3) and osteochondral splitting (0-3) were performed as previously described by McIlwraith et al. 2010 using hematoxylin & eosin (H&E) staining.

The native and biglycan neoepitope stained tissues were imaged at 200× magnification using bright field microscope. Stained area was quantified in photomicrographs (Non loading area cartilage, radial facet bone+cartilage, loading area−bone+cartilage) using Fiji Image J program (ImageJ, National Institute of health Bethesda, MD). The data is expressed as fold change compared to the control group.

Immunohistochemistry staining of cartilage and bone with a polyclonal antibody towards native molecule of biglycan show mostly extracellular staining of the cartilage and bone matrix. However, immunohistochemistry staining of cartilage and bone with a polyclonal antibody towards biglycan neo-epitope show intracellular staining of cells in cartilage and bone.

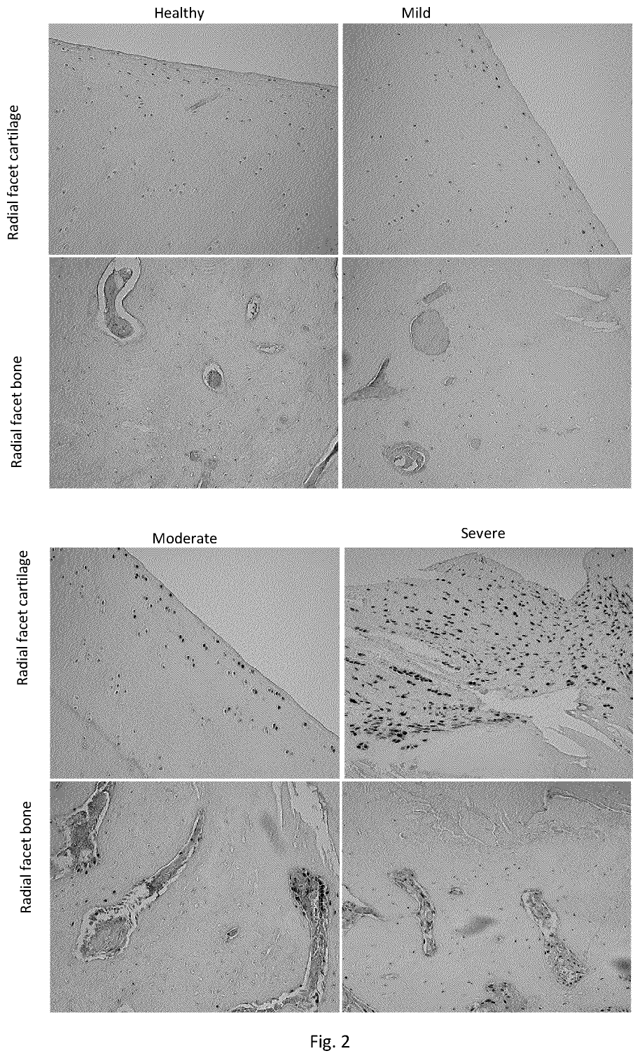

The presence of native biglycan and biglycan neoepitope in cartilage bone tissue sections in a horse with normal. Mild, moderate and severe cartilage and bone lesions associated with OA (FIG. 2).

### Example 4

The presence of the neoepitope (N-terminal-GLGHN) of biglycan was evaluated in horse serum, synovial fluid and cartilage-bone tissues using the ELISA from example 2.

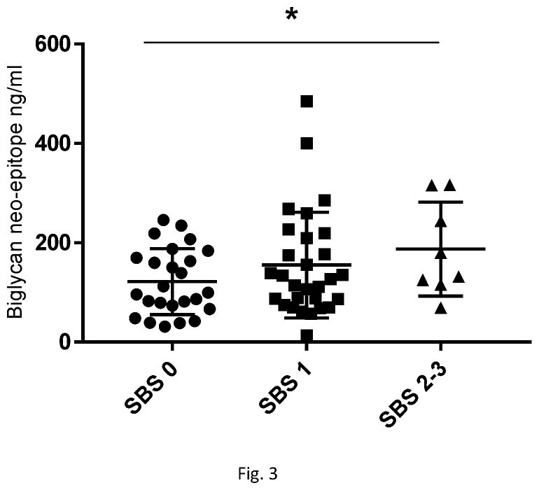

Concentration of the biglycan neopitope in synovial fluid from horses with OA in the carpal joint correlated to the increased radiographic subchondral bone sclerosis (SCBS) of the third carpal bone (FIG. 3).

### Example 5

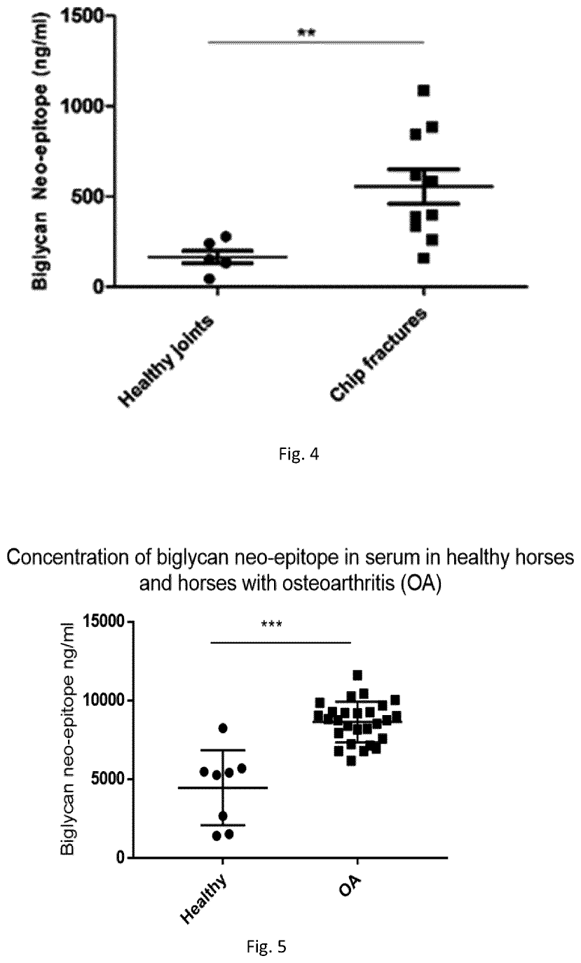

A dramatic increase of the biglycan neoepitope was found in synovial fluid from the mid carpal joint with chip fractures compared to normal joints (FIG. 4).

### Example 6

Concentration of biglycan neo-epitope in serum shows an increased concentration of the biglycan neoepitope in horses with osteoarthritis compared to healthy horses (FIG. 5).

### Example 7

Saliva was collected from horses using Equisal saliva collection kit (Purchased: Austindavis biologics ltd), inserting the swab through the interdental space on the horse tongue until the volume indicator changed colour. Approximately 500 ul of crude saliva was sampled with the device. The samples were stabilised in 2 ml preservative buffer (1×PBS (sodium chloride 137 mM, potassium chloride 2.7 mM, disodium phosphate 11.9 mM), 0.05% tween 20, 0.05% bromonitrodioxane and 0.05% sodium azide), temporarily stored at −20° C. before being thawed and centrifuged at 3000 g for 5 min. All samples were aliquoted in a minimum volume of 500 μl and stored at −80° C. prior to analysis.

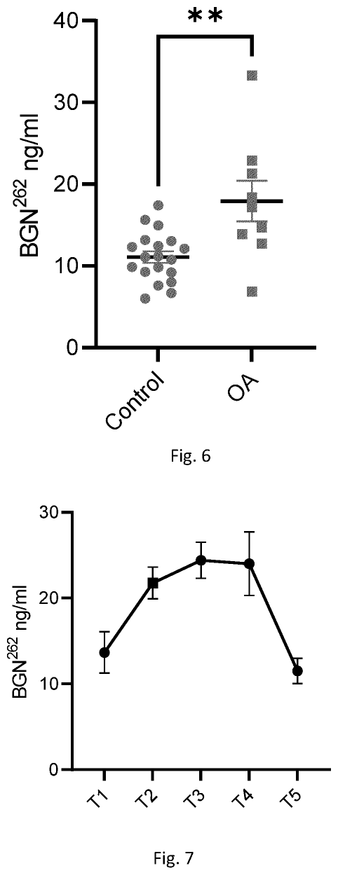

Presence of the biglycan neoepitope in the saliva samples was determined using the custom made ELISA described above. The results are shown in FIG. 6. The concentration of the BGN²⁶² increased in in OA horses with radiographic changes vs control horses (p=0.01962 with t-test and p=0.0196 with Wilcoxon).

### Example 8

Healthy horses were allowed to undergo warmup for 20 minutes (jogging trot) and exercise for 20 minutes (strenuous training). Saliva was sampled from the horses at the following time points.
Time point 1=1 hour pre-exercise (sampled in the stable)
Time point 2=after 20 minutes of warm up
Time point 3=after 20 minutes of exercise
Time point 4=after 10 minutes of cool down
Time point 5=1 hour post-exercise (sampled in the stable)

The presence of the biglycan neoepitope was determined using ELISA as described above.

The results are shown in FIG. 7. The concentration of the BGN²⁶² show a significant increase in saliva from horses that were exercised (timepoint 3, (T3) compared to timepoint at rest timepoint (TP) 1 (p=0.001).

### Example 9

Presence of the biglycan neo-epitope in serum samples from colon cancer patients before and after removal of the primary tumor was determined using the custom made ELISA described above.

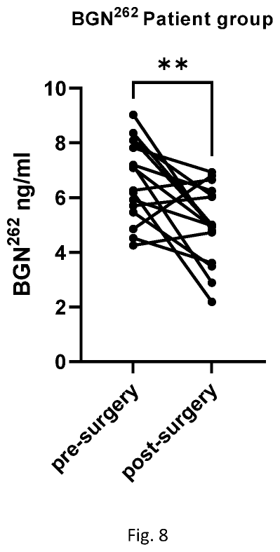

The results are shown in FIG. 8. The concentration of the BGN²⁶² peptide showed a significant decrease in serum after colon cancer surgery in patients (p=0.001). The data show that the level of biglycan neoepitope decrease after removal of the primary tumor suggesting that the primary tumor may cause the degradation of biglycan. Biglycan neoepitope levels may be used to track the invasiveness of the tumor.

### Example 10

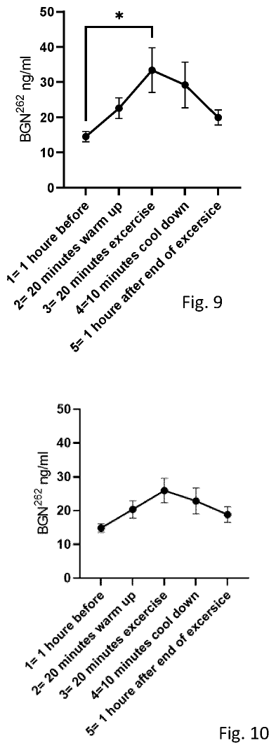

In a cross sectional study, saliva was sampled from nine horses exercised on two different footings in a crossover design. Statistically significant increase in BGN 262 levels at time point 3 was found in horses exercised on fibre-sand footing (FIG. 9). The levels did not change when horses were exercised on sand (FIG. 10).

### Example 11

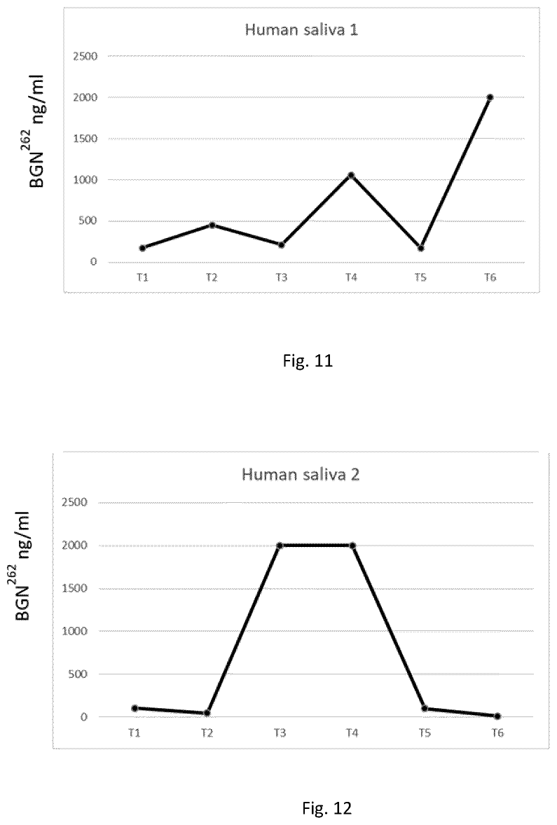

The concentration of the BGN²⁶² in saliva from humans that were exercised (time point 4, (T4) increased compared to time point at rest time point (TP). At time point 5 and 6 the concentration is reduced as the joint is no longer exposed to elevated dynamic compression. Time point 1=1 hour pre-exercise, Time point 2=direct before exercise, Time point 3=after 10 minutes of walking, Time point 4=after 10 minutes running on a treadmill, Time point 5=after 10 minutes cool down (walking), Time point 6=1 hour post-exercised. FIG. 11 display BGN²⁶² in saliva from an older person not used to running. The graph shows a peak at T4 after running. At T6 a second peak is observed which results from catabolic activity produced during exercise. FIG. 12 shows BGN²⁶² in saliva from a young person used to running and the elevation observed already at T3 and T4 is similar to the trained horses. No additional peaks are found possibly due to no catabolic degradation of biglycan.

1. A method of diagnosis of a disease selected from the group consisting of: bone sclerosis, fractures, chip fractures of the joint, avulsion fractures, bone bruise, osteoporosis, cancer, and rheumatoid arthritis, comprising providing a sample previously isolated from a subject and analysing the sample for presence of a peptide comprising the amino acid sequence N-terminal-GLGHN in the sample.

2. The method of claim 1 where the disease is selected from the group consisting of: bone sclerosis, fractures, chip fractures of the joint, avulsion fractures, bone bruise, or osteoporosis and colon cancer.

3. The method of claim 1 where the disease is selected from the group consisting of: bone sclerosis, fractures, chip fractures of the joint, avulsion fractures and bone bruise.

4. The method of claim 1 where the cancer is colon cancer.

5. The method of claim 1 where the sample is a sample of synovial fluid, spinal fluid (liquor), serum, blood, blood plasma, urine or saliva.

6. The method of claim 5 where the sample is a sample of saliva.

7. An antibody that specifically binds to a peptide comprising the amino acid sequence N-terminal-GLGHN (SEQ ID NO 1) where the antibody binds to the sequence N-terminal GLGHN (SEQ ID NO 1).

8. The antibody according to claim 7 for use in diagnosis of a disease selected from the group consisting of: bone sclerosis, fractures, chip fractures of the joint, avulsion fractures, bone bruise, osteoporosis, cancer and rheumatoid arthritis, in a subject.

9. The antibody for use according to claim 7 where the diagnosis is diagnosis of a disease selected from the group consisting of: bone sclerosis, fractures, chip fractures of the joint, avulsion fractures bone bruise and osteoporosis.

10. The antibody for use according to claim 7 where the antibody is used for detecting the amount of a peptide comprising the amino acid sequence N-terminal-GLGHN in a sample from a subject.

11. The antibody for use according to claim 10 where the sample is selected from the group consisting of: a sample of synovial fluid, spinal fluid (liquor), serum, blood, blood plasma, urine and saliva.

12. A method for treating a disease selected from the group consisting of: osteoarthritis, bone sclerosis, fractures, chip fractures of the joint and avulsion fractures in a subject comprising the steps of:
a) repeatedly obtaining samples from the subject and analysing the presence of a peptide comprising the amino acid sequence N-terminal-GLGHN (SEQ ID NO 1) in the samples; and
b) if the level of peptide in the subject in a sample is above a predetermined level, determining that the subject should be treated, where the treatment is resting the subject.

13. A kit comprising an antibody according to claim 7.

14. The kit according to claim 13 where the antibody is comprised in a diagnostic device for single use, where the kit additionally comprises a saliva sampling device.

15. The kit according to claim 14 where the diagnostic device is a lateral flow device.

16. A peptide comprising the amino acid sequence N-terminal-GLGHN (SEQ ID NO 1), said peptide having a length of from 5 to 20 residues.

17. Use of a peptide comprising an amino acid sequence N-terminal-GLGHN (SEQ ID NO 1) according to claim 16 for the production of an antibody.

18. A method for determining the properties of a footing for a horse arena, comprising obtaining a sample from a horse that has been exercising on the footing and analysing the sample for presence of a peptide comprising the amino acid sequence N-terminal-GLGHN according to claim 16 in the sample.
